(19) United States
(12) Patent Application Publication
(10) Pub. No.: US 2021/0391090 A1
(43) Pub. Date: Dec. 16, 2021

(54) Classification of Immuno-Oncology Impact

(71) Applicant: ASTRAZENECA AB, Sodertalje (SE)

(72) Inventors: Nicole Denise St. Jean, Gaithersburg, MD (US); Craig Russell Maxwell, Gaithersburg, MD (US); Elizabeth Maybelle Shaheen-Dumke, Gaithersburg, MD (US); Evangelos Pazarentzos, Gaithersburg, MD (US); Sesquile Ramon, Gaithersburg, MD (US)

(21) Appl. No.: 17/337,099

(22) Filed: Jun. 2, 2021

Related U.S. Application Data
(60) Provisional application No. 63/039,221, filed on Jun. 15, 2020.

(51) Int. Cl. G16H 70/60

(52) U.S. Cl. CPC G16H 70/60

(57) ABSTRACT

An embodiment may involve reading conditions for respectively determining research impacts of cancer-related journal publications, congress activities, guideline activities, and trial activities; reading data regarding the cancer-related journal publications, the congress activities, the guideline activities, and the trial activities of each of a plurality of individuals; applying the conditions to the data on a per-individual basis to determine partial grades and overall grades for each of the plurality of individuals, wherein applying the conditions to the data of a particular individual involves: determining individual partial grades respectively for the cancer-related journal publications, the congress activities, the guideline activities, and the trial activities of the particular individual, and determining an individual overall grade for the particular individual based on a sum of the individual partial grades; and storing the overall grades and the partial grades for each of the plurality of individuals.

### CROSS-REFERENCE TO RELATED APPLICATION

This application claims priority to U.S. provisional patent application No. 63/039,221, filed Jun. 15, 2020, which is hereby incorporated by reference in its entirety.

### BACKGROUND

In immuno-oncology and other disciplines, it is desirable to be able to identify individuals with specific expertise in particular sub-fields of such a discipline. But doing so in an objective and timely fashion has proven challenging. There are tens of thousands of potential experts, each with varying degrees of academic and scientific accolades and recognition. All too often, experts are identified based on word-of-mouth or personal relationships, rather than objective data. But if such data is used to identify experts, the amount of data available can be overwhelming and extremely time-consuming to filter. As a result, it is challenging to find a quality signal of who are the “top experts” within a vast corpus of noise. And, with the scientific and medical professions changing at a rapid rate, by the time that the full set of data is thoroughly analyzed, the results obtained may already be out of date.

### SUMMARY

The embodiments herein address these and other problems by providing an objective and rigorous and tailored data-mining technique for identifying expertise in a given discipline (i.e. lung cancer and liver cancer). While the discipline considered in the following example embodiments is immuno-oncology, the techniques described by these embodiments could be used with other disciplines.

In particular, these techniques involve calculating an expertise score (grade) for an individual based on publications authored or coauthored by the individual, congresses (conferences) at which the individual presented or had a role, published guidelines with which the individual was involved, clinical trials in which the individual was involved, journal editorial board positions held by the individual, and/or organizations in which the individual had a role. The score takes into account not only the volume of such activities and engagements involving the individual, but also the impact of these engagements and the individual's role in the activities and engagements, among other possible factors. As a consequence, an objective assessment of an individual's expertise can be determined.

This is a departure from past techniques that were based on more simplistic and non-objective measures such as publication count based on volume only or personal relationships. Further, with the number of potential experts being in the tens of thousands the amount of time required to gather this information and perform the calculations introduced herein would be prohibitive. Manually carrying out such tasks would take months, and the results thereof could be obsolete by the time they were determined. Therefore, by performing these calculations by way of a computing system allows the results to be produced in a reduced time frame of hours or days, guaranteeing that these results more accurately reflect the expertise of individuals at any point in time.

These assessments of expertise are remarkably useful to many organizations. For example, a pharmaceutical, biotechnology, academic institution, or other organization developing immunotherapies may be ready to initiate a clinical trial, but would greatly benefit from contemporary and topical experts serving as advisors for the trial. With the embodiments herein, such experts can be identified objectively, thereby increasing the efficacy of such trials. Other benefits are possible as well.

Accordingly, a first example embodiment may involve reading, by one or more processors and from persistent storage, conditions for respectively determining research impacts of cancer-related journal publications, congress activities, guideline activities, and clinical trial activities. The first example embodiment may also involve reading, by the one or more processors and from the persistent storage, data regarding the cancer-related journal publications, the congress activities, the guideline activities, and the trial activities of each of a plurality of individuals. The first example embodiment may also involve applying, by the one or more processors, the conditions to the data on a per-individual basis to determine partial grades and overall grades for each of the plurality of individuals, wherein applying the conditions to the data of a particular individual involves: determining individual partial grades respectively for the cancer-related journal publications, the congress activities, the guideline activities, and the trial activities of the particular individual, and determining an individual overall grade for the particular individual based on a sum of the individual partial grades. The first example embodiment may also involve writing, by the one or more processors and to the persistent storage, the overall grades and the partial grades for each of the plurality of individuals.

In a second example embodiment, an article of manufacture may include a non-transitory computer-readable medium, having stored thereon program instructions that, upon execution by a computing system, cause the computing system to perform operations in accordance with the first example embodiment.

In a third example embodiment, a computing system may include at least one processor, as well as memory and program instructions. The program instructions may be stored in the memory, and upon execution by the at least one processor, cause the computing system to perform operations in accordance with the first example embodiment.

In a fourth example embodiment, a system may include various means for carrying out each of the operations of the first example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 illustrates a schematic drawing of a computing device, in accordance with example embodiments.

FIG. 2 illustrates a schematic drawing of a server device cluster, in accordance with example embodiments.

FIG. 3 illustrates techniques for evaluating expertise based on a set of factors, in accordance with example embodiments.

FIG. 4A provides conditions for evaluating the impact of publications relating to liver cancer, in accordance with example embodiments.

FIGS. 4B and 4C provide evaluations of journals based on the conditions of FIG. 4A, in accordance with example embodiments.

FIG. 5A provides conditions for evaluating the impact of congresses relating to liver cancer, in accordance with example embodiments.

FIG. 5B provides evaluations of congresses based on the conditions of FIG. 5A, in accordance with example embodiments.

FIG. 6A provides conditions for evaluating the impact of guidelines relating to liver cancer, in accordance with example embodiments.

FIG. 6B provides evaluations of guidelines based on the conditions of FIG. 6A, in accordance with example embodiments.

FIG. 7 provides conditions for evaluating the impact of clinical trials relating to liver cancer, in accordance with example embodiments.

FIG. 8A provides conditions for evaluating the impact of journal editorial boards relating to liver cancer, in accordance with example embodiments.

FIG. 8B provides evaluations of journal editorial boards based on the conditions of FIG. 8A, in accordance with example embodiments.

FIG. 9A provides conditions for evaluating the impact of organizations relating to liver cancer, in accordance with example embodiments.

FIG. 9B provides evaluations of organizations based on the conditions of FIG. 9A, in accordance with example embodiments.

FIG. 10A provides conditions for evaluating the impact of publications relating to lung cancer, in accordance with example embodiments.

FIGS. 10B and 10C provide evaluations of journals based on the conditions of FIG. 10A, in accordance with example embodiments.

FIG. 11A provides conditions for evaluating the impact of congresses relating to lung cancer, in accordance with example embodiments.

FIG. 11B provides evaluations of congresses based on the conditions of FIG. 11A, in accordance with example embodiments.

FIG. 12A provides conditions for evaluating the impact of guidelines relating to lung cancer, in accordance with example embodiments.

FIG. 12B provides evaluations of guidelines based on the conditions of FIG. 12A, in accordance with example embodiments.

FIG. 13 provides conditions for evaluating the impact of clinical trials relating to liver cancer, in accordance with example embodiments.

FIG. 14A provides conditions for evaluating the impact of journal editorial boards relating to lung cancer, in accordance with example embodiments.

FIG. 14B provides evaluations of journal editorial boards based on the conditions of FIG. 14A, in accordance with example embodiments.

FIG. 15A provides conditions for evaluating the impact of organizations relating to lung cancer, in accordance with example embodiments.

FIG. 15B provides evaluations of organizations based on the conditions of FIG. 15A, in accordance with example embodiments.

FIG. 16 illustrates uses of the conditions and evaluations, in accordance with example embodiments.

FIG. 17 is a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. Example Computing Devices and Cloud-Based Computing Environments

The embodiments herein may involve the use of various types of computing systems and environments. The following embodiments describe architectural and functional aspects of example computing systems, as well as the features and advantages thereof.

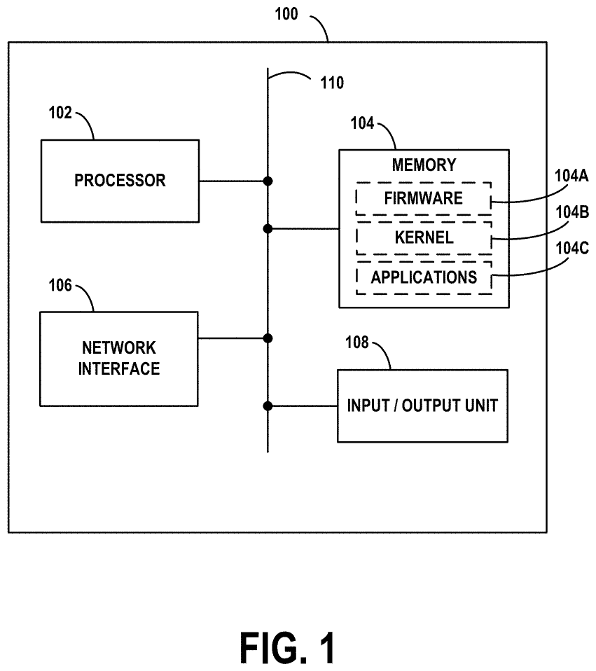

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and an input/output unit 108, all of which may be coupled by a system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently-used instructions and data.

Memory 104 may be any form of computer-usable memory, including but not limited to random access memory (RAM), read-only memory (ROM), and non-volatile memory (e.g., flash memory, hard disk drives, solid-state drives, compact discs (CDs), digital video discs (DVDs), and/or tape storage). Thus, memory 104 represents both main memory units, as well as long-term storage. Other types of memory may include biological memory.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling and management of processes, input / output, and communication. Kernel 104B may also include device drivers that allow the operating system to communicate with the hardware modules (e.g., memory units, networking interfaces, ports, and busses), of computing device 100. Applications 104C may be one or more user-space software programs, such as web browsers or email clients, as well as any software libraries used by these programs. Memory 104 may also store data used by these and other programs and applications.

Network interface 106 may take the form of one or more wireline interfaces, such as Ethernet (e.g., Fast Ethernet, Gigabit Ethernet, and so on). Network interface 106 may also support communication over one or more non-Ethernet media, such as coaxial cables or power lines, or over wide-area media, such as Synchronous Optical Networking (SONET) or digital subscriber line (DSL) technologies. Network interface 106 may additionally take the form of one or more wireless interfaces, such as IEEE 802.11 (Wifi), BLUETOOTH®, global positioning system (GPS), or a wide-area wireless interface. However, other forms of physical layer interfaces and other types of standard or proprietary communication protocols may be used over network interface 106. Furthermore, network interface 106 may comprise multiple physical interfaces. For instance, some embodiments of computing device 100 may include Ethernet, BLUETOOTH®, and Wifi interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

In some embodiments, one or more computing devices like computing device 100 may be deployed to support a server-based architecture. The exact physical location, connectivity, and configuration of these computing devices may be unknown and/or unimportant to client devices. Accordingly, the computing devices may be referred to as “cloud-based” devices that may be housed at various remote data center locations.

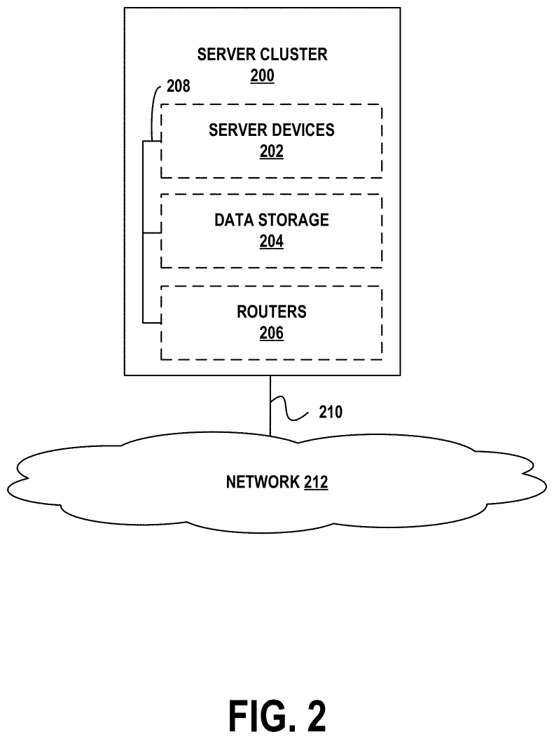

FIG. 2 depicts a cloud-based server cluster 200 in accordance with example embodiments. In FIG. 2, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purpose of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid-state drives. The drive array controllers, alone or in conjunction with server devices 202, may also be configured to manage backup or redundant copies of the data stored in data storage 204 to protect against drive failures or other types of failures that prevent one or more of server devices 202 from accessing units of data storage 204. Other types of memory aside from drives may be used.

Routers 206 may include networking equipment configured to provide internal and external communications for server cluster 200. For example, routers 206 may include one or more packet-switching and/or routing devices (including switches and/or gateways) configured to provide (i) network communications between server devices 202 and data storage 204 via local cluster network 208, and/or (ii) network communications between the server cluster 200 and other devices via communication link 210 to network 212.

Additionally, the configuration of routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency and/or other design goals of the system architecture.

As a possible example, data storage 204 may include any form of database, such as a structured query language (SQL) database. Various types of data structures may store the information in such a database, including but not limited to tables, arrays, lists, trees, and tuples. Furthermore, any databases in data storage 204 may be monolithic or distributed across multiple physical devices. The information stored in such a database (i.e., the information regarding potential experts described herein) may be updated in real time or on a periodic or irregular basis.

Server devices 202 may be configured to transmit data to and receive data from data storage 204. This transmission and retrieval may take the form of SQL queries or other types of database queries, and the output of such queries, respectively. Additional text, images, video, and/or audio may be included as well. Furthermore, server devices 202 may organize the received data into web page representations. Such a representation may take the form of a markup language, such as the hypertext markup language (HTML), the extensible markup language (XML), or some other standardized or proprietary format. Moreover, server devices 202 may have the capability of executing various types of computerized scripting languages, such as but not limited to Perl, Python, PHP Hypertext Preprocessor (PHP), Active Server Pages (ASP), JAVASCRIPT®, and so on. Computer program code written in these languages may facilitate the providing of web pages to client devices, as well as client device interaction with the web pages.

### II. Expertise Assessment and General Framework

As noted above, expertise assessment can be a key aspect of being able to successfully carry out a meaningful clinical trial. Other areas in which an expert can improve the outcome of medical research and development include drug discovery, pre-clinical research, navigating regulatory requirements, pharmaceutical synthesis, trial outcome evaluation, and so on.

The embodiments herein are designed to identify the most active and impactful experts in immuno-oncology and other cancer-related fields by using key activity indicators. These experts can then be invited to participate in research and development activities. Doing so is likely to produce a better outcome for these activities, especially for organizations that lack expertise in key areas.

While the examples below provide techniques and data sources for identifying experts in immuno-oncology disciplines relating to liver and lung cancer, other techniques and data sources could be incorporated into these embodiments to identify experts in other disciplines. For example, these techniques could be used to identify experts in other types of medical sciences, as well as psychology, engineering, management, and additional disciplines.

Regardless, individuals can be evaluated for expertise based on the following activities: publications authored or coauthored by the individual, congresses (conferences) at which the individual presented or had a key role, published guidelines with which the individual was involved, clinical trials in which the individual was involved, journal editorial board positions held by the individual, and/or organizations in which the individual had a key role. A sub-score is determined for each of these activities, and then the sub-scores are combined to produce a final score. In these embodiments, sub-scores may be referred to as partial grades, while final scores may be referred to as grades or overall grades.

The sub-scores are designed to reflect impact and relevance. As a result, more impactful and relevant activity produces a higher sub-score than less impactful or relevant activity. Impact may be based on the prestige, selectiveness, and/or agreed-upon quality of the activity. Relevance may be based on how closely aligned the activity is to the expertise sought.

The examples herein use sub-scores with ranges of 0-30 per activity. For a particular individual, each journal publication, congress activity, guideline activity, clinical trial activity, journal editorial board activity, and organization activity is assigned a sub-score from 0-30. These sub-scores can be summed on a per activity basis to determine sub-scores for each category of publication, congress, guideline, clinical trial, journal editorial board and organization. A per-activity average of these sub-scores may be taken to map the sub-scores back into the 0-30 range.

A sub-score of 0 would indicate that the particular individual had no involvement in that activity—i.e., a sub-score of 0 in congress activity may be interpreted as the particular individual not being involved in any significant fashion with congresses. Another example is that a journal publications sub-score of 0 would indicate that the particular individual is not listed as an author in the data set. This would be highly unlikely, as experts in any given field tend to publish journal articles relatively frequently.

These average sub-scores may be summed to determine the final score. Thus, the final score has a range of 0 to a maximum of 180. Nonetheless, other ranges of sub-scores and/or final scores could be used. The higher the final score, the more expertise the individual has with respect to the areas of relevance. Through this methodology, a list of top experts can be rapidly and objectively identified.

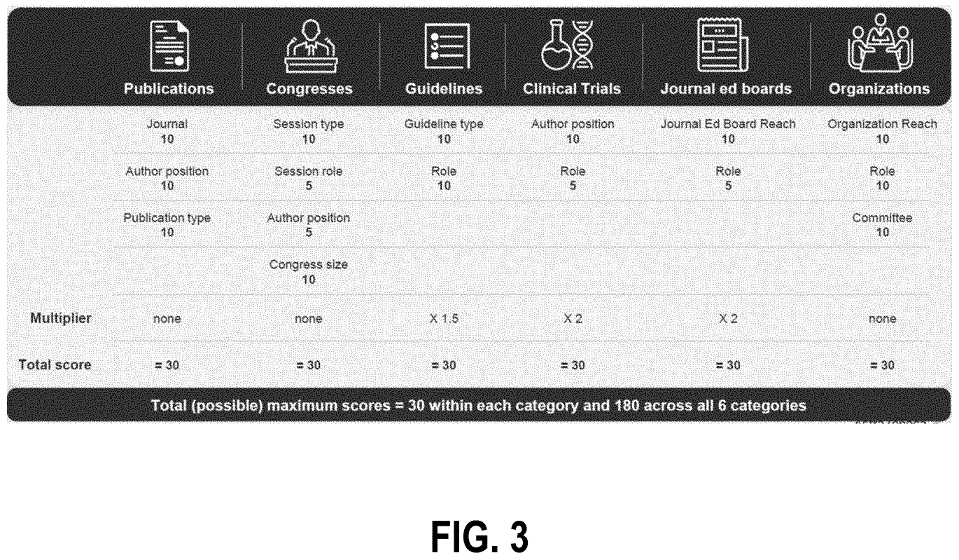

FIG. 3 provides an example of how values and weights can be allocated to the activities. More detailed definitions of these activities are provided below, but FIG. 3 serves as a high-level overview of the scoring system.

### A. Publications

In academia, journal publications are one of the most important and prestigious outlets of an individual's research. But not all journals have the same degree of selectiveness or relevance to the discipline of interest. Therefore, different journal publications should be evaluated in different manners. Thus, for the publications sub-score, 10 points are allocated based on the journal in which the publication appears, another 10 points are allocated based on the position of the individual in the list of authors, and the final 10 points are allocated based on the type of publication.

Points may be assigned to each journal that publishes articles related to the discipline of interest. For example, a highly-selective and relevant journal may be assigned 10 points, whereas a less prestigious or less relevant journal might be assigned fewer points.

Points may be assigned based on author position with preference given to first and last authors over second and other (middle) authors. For instance, the most points may be assigned to an individual who is a last author, as the last author position is often given to a principal investigator who is leading a research team. The second most points may be assigned to an individual who is a first author, as the first author position is often given to a person who contributed the most to the research and/or leading the writing of the paper. Middle authors may be assigned fewer points than either the last author or the first author. For publications where there is only one author, that individual may be given the full amount of points (e.g., 10). As noted above, the lowest sub-score for a listed author is 5 points.

Points may be assigned to publication type based on the extent and nature of the publication. For example, in the medical sciences fields, a clinical research paper may be assigned the most points, while review articles, editorials, congress papers, letters and comments may be assigned decreasing numbers of points.

### B. Congresses (Conferences)

Congresses and conferences are regular or semi-regular gatherings of large numbers of individuals within one or more disciplines. Many formal presentations are made at congresses and conferences, including those related to papers, posters, tutorials, debates, etc. The type and manner of the presentations may be more or less impactful. For the congress sub-score, 10 points are allocated based on the type of session at which the individual presented, 5 points are allocated based on the individual's role in that session, another 5 points are allocated based on the position of the individual in the list of authors, and a further 10 points are allocated based on the size and relevance of the congress. In this disclosure, no distinction will be made between congresses and conferences.

Points may be assigned to the types of session based on its prominence or prestige. For example, a keynote or plenary speaking session may be assigned 10 points, while progressively fewer points may be assigned to other presentations, sponsored sessions, debates, and workshops, with fewest points assigned to poster sessions.

Points may also be assigned to the individual's role in a session based on the prominence of that role. For example, session chairs or moderators may be assigned more points than speakers or panelists.

For presentations, papers, and/or posters in a congress, points may be assigned based on author position with preference given to first, second, and last authors over other (middle) authors. These point assignments may be similar to those discussed above for publications.

Points may further be assigned based on the size and relevance of the congress. These assignments may vary from discipline to discipline. But in general, the more individuals that attend the congress and the more focused the congress is on the discipline of interest, the more points it is assigned.

### C. Guidelines

A medical guideline is a specific type of publication that provides recommendations and conditions regarding diagnosis, management, and treatment of specific medical conditions. Guidelines are often published by respected governmental or private organizations with the goal of capturing the state-of-the-art understanding for the topics therein. For the guidelines sub-score, 10 points are allocated based on the guideline type, and another 10 points are allocated based on the individual's role in development of the guideline. The sum of these two amounts is multiplied by 1.5 to produce a sub-score in the range of 0-30.

The guideline type may be based on the organization that publishes the guideline, the prestige of that organization, and the relevance of the guideline's topics to that of the discipline of interest. More points may be assigned to prestigious organizations with such relevance.

The individual's role in development of the guideline may be assigned points based on its importance. For example, a lead role may be given more weight than that of a non-lead author or reviewer.

### D. Clinical Trials

For the clinical trials sub-score, 10 points are allocated based on the position of the individual in the list of authors, and 5 points are allocated based on the individual's role in the clinical trial. The sum of these two amounts is multiplied by 2 to produce a sub-score in the range of 0-30.

As was the case for authorship of journal and congress papers, points may be assigned based on author position with preference given to first and last authors over second and other (middle) authors. These point assignments may be similar to those discussed above for publications. Points assigned based on the individual's role may be arranged to assign more points to a principal investigator than a study director or investigator, for example.

### E. Journal Editorial Boards

Serving on a journal editorial board is often considered to be an esteemed position, but the amount of prestige afforded can vary. For the journal editorial boards sub-score, 10 points are allocated based on the reach of the board on which the individual serves, and 5 points are allocated based on the individual's role on the board. The sum of these two amounts is multiplied by 2 to produce a sub-score in the range of 0-30.

The reach of a journal editorial board may be based on the yearly average number of citations that recent articles published in the journal has received and or the average citations per article published in the journal over a multi-year period (e.g., 2 years, 3, year, 5 years). The individual's role on the board may be given more weight if the individual is a chair or president, and decreasing amounts if the individual is a secretary or treasurer, reviewer, a past board member, or an advisor or consultant.

### F. Organizations

Membership in various organizations are also indicative of an individual's expertise. For the organizations sub-score, 10 points are allocated based on the reach of the organization and its relevance to the discipline of interest, another 10 points are allocated based on the role of the individual within the organization, and another 10 points are allocated based on the committee(s) within the organization in which the individual participates.

The reach of the organization is based on the membership size of the organization, with more points being assigned to organizations with larger reaches and focused on the discipline of interest. The individual's role within the organization may be given more weight if the individual is a chair or president, and decreasing amounts if the individual is a past board member, a secretary or treasurer, an advisor or consultant, reviewer, or member. The committee(s) that the individual serves on may be given more weight based on their prestige and leadership roles within the organization, and less weight if they are more administrative.

### III. Example: Hepatocellular Carcinoma

In order to illustrate these embodiments, specific examples are provided in the sub-sections below for hepatocellular carcinoma (HCC), a common form of liver cancer. Thus, the values assigned to various publications, congresses, guidelines, clinical trials, journal editorial boards, and organizations are based on their prominence and relevance to the study and treatment of this form of liver cancer. In all of the point assignments below, exactly one point value from each table is selected. If the individual qualifies to be allocated different point values from the same table, the most accurate description of the individual's activity determines the point value. Failing that, the highest or lowest such point value may be selected based on a pre-defined configuration.

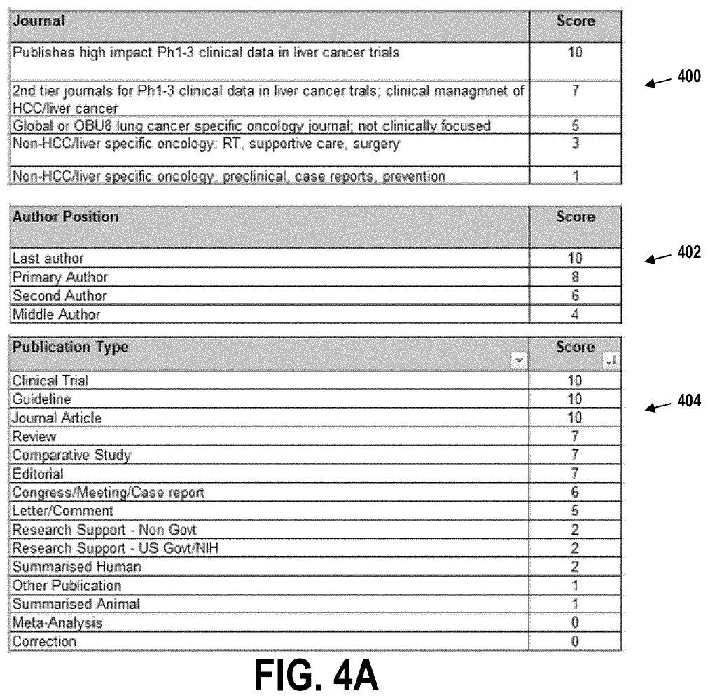

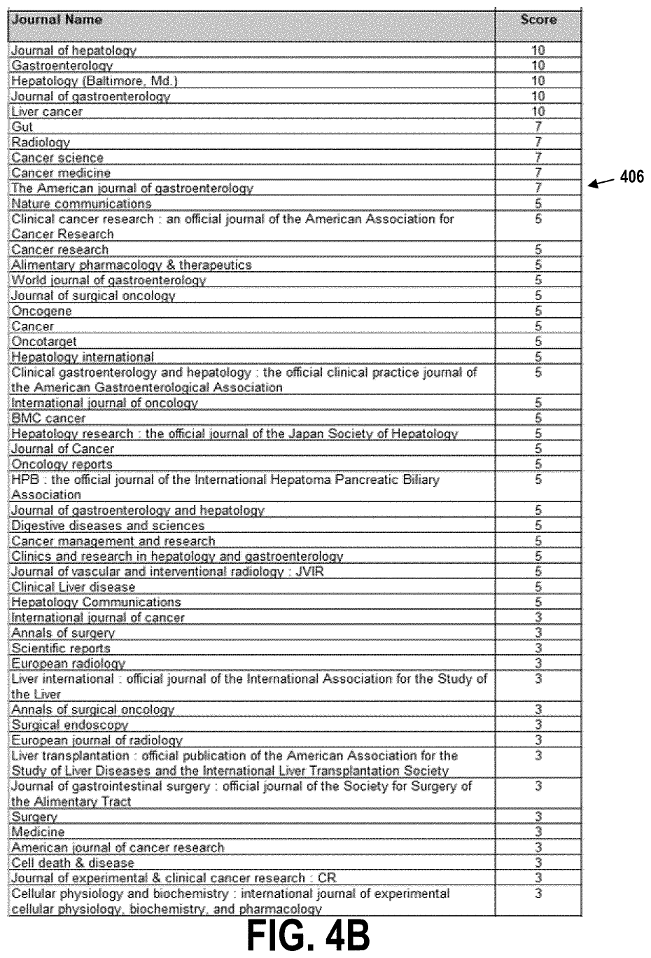

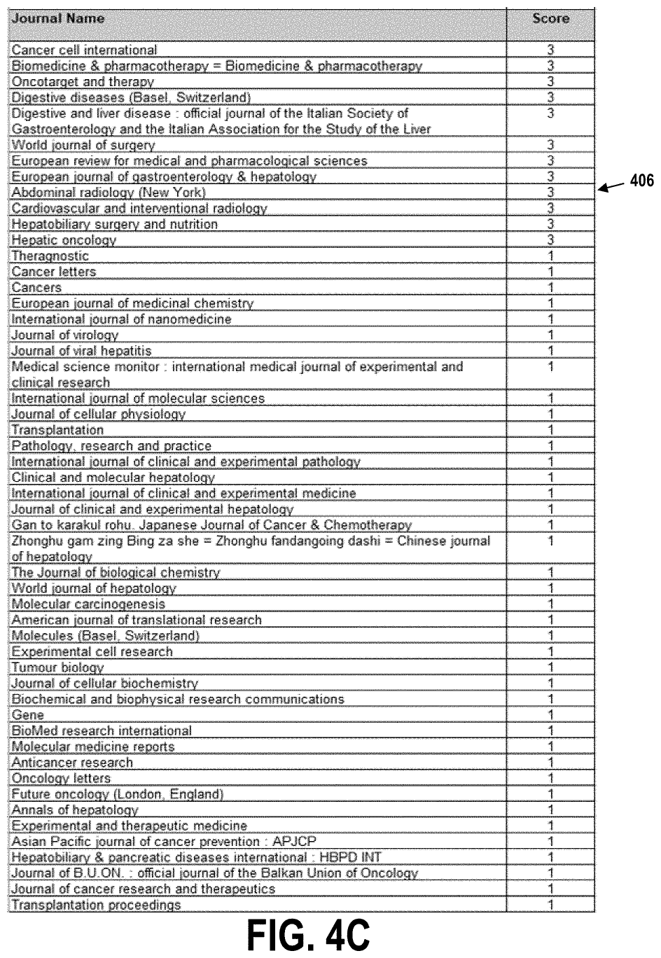

FIG. 4A provides assignments of points to an individual's journal publications related to HCC. For journals, and as shown in table 400, 10 points are assigned to journals that publish high impact phase 1-3 clinical data in liver cancer trials, 7 points are assigned to second-tier journals that publish phase 1-3 clinical data in liver cancer trials and journals focused on clinical management of HCC and/or liver cancer, 5 points are assigned to global or liver cancer oncology journals in the US, Italy, Spain, UK, Germany, France, China or Japan regions that are not clinically focused, 3 points are assigned to non-HCC and non-liver oncology journals focused on radiation therapy, supportive care, or surgery, and 1 point is assigned to non-HCC and non-liver oncology journals focused on preclinical studies, case reports, or prevention.

For author position, and as shown in table 402, 10 points are assigned to the last author, 8 points are assigned to the primary author, 6 points are assigned to the second author, and 4 points are assigned to the middle author(s). Here, a “middle author” is any author positioned in other than the first, second, or last position. For example, on a paper with five authors, the authors in the third and fourth positions are both middle authors.

For publication type, and as shown in table 404, 10 points are assigned to papers on clinical trials, papers on guidelines, and journal articles. Other types of publications are assigned increasingly fewer points. For example, meta-analyses and corrections are assigned 0 points. Note that, to the extent that these journal articles overlap with those in the journal publications category, the double counting of publications is positive as it highlights high-impact trials in this space. In practice, the amount of overlap is low and does not have a significant impact on the overall score in most cases.

The lowest score for a listed author is 5 points. This occurs when the author is a middle author (4 points), the journal is in the non-HCC and non-liver oncology category (1 point), and the type of publication is a meta-analysis or correction (0 points).

FIGS. 4B and 4C provide table 406, which maps actual journals to assigned point values, in accordance with table 400. As indicated by the conditions of table 400, the journals assigned 10 points are assigned to prestigious journals that focus on liver cancer, while increasingly fewer points are assigned to journals that are less prestigious or are less focused on liver cancer.

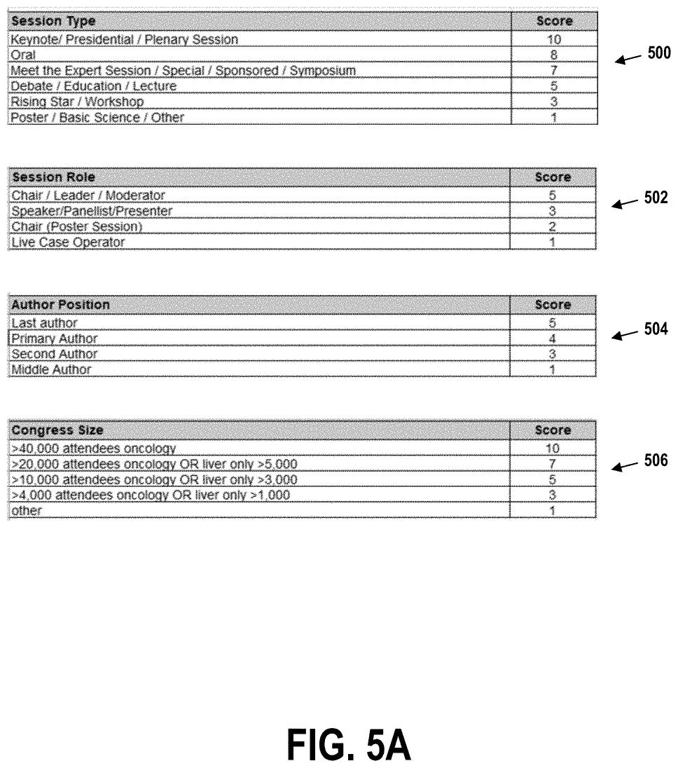

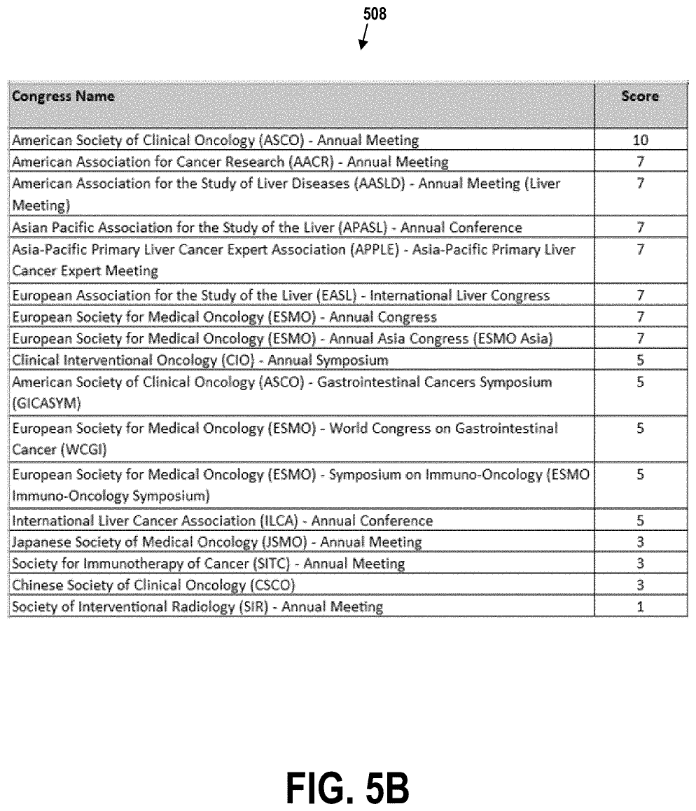

FIG. 5A provides assignments of points to an individual's congress activities related to HCC. For the session type, and as shown in table 500, keynote, presidential and plenary sessions are assigned 10 points, other oral presentations are assigned 8 points, meet the experts sessions, special sessions, sponsored sessions, and symposia are assigned 7 points, debates, educational sessions, and lectures are assigned 5 points, rising star sessions and workshops are assigned 3 points, and posters, basic science sessions other types of sessions are assigned 1 point.

For the individual's role within the session, and as shown in table 502, chairs, leaders, and moderators are assigned 5 points, speakers, panelists, and presenters and assigned 3 points, chairs of poster sessions are assigned 2 points, and live case operators are assigned 1 point.

For author position, and as shown in table 504, 5 points are assigned to the last author, 4 points are assigned to the primary author, 3 points are assigned to the second author, and 1 point is assigned to the middle author(s). Here, the term “middle author” is defined as was the case for journal publications.

For congress size, and as shown in table 506, congresses with over 40,000 attendees and focused on oncology are assigned 10 points, congresses with over 20,000 attendees (and up to 40,000 attendees) focused on oncology or congresses with over 5,000 attendees focused on liver cancer are assigned 7 points, congresses with over 10,000 attendees (and up to 20,000 attendees) focused on oncology or congresses with over 3,000 attendees (and up to 5,000 attendees) focused on liver cancer are assigned 5 points, congresses with over 4,000 attendees (and up to 10,000 attendees) focused on oncology or congresses with over 1,000 attendees (and up to 3,000 attendees) focused on liver cancer are assigned 3 points, and other congresses are assigned 1 point.

FIG. 5B provides table 508, which maps actual congresses to assigned point values, in accordance with table 506. As indicated by the conditions of table 506, larger congresses that are more focused on liver cancer are allocated more points, while increasingly fewer points are assigned to congresses that are smaller or are less focused on liver cancer.

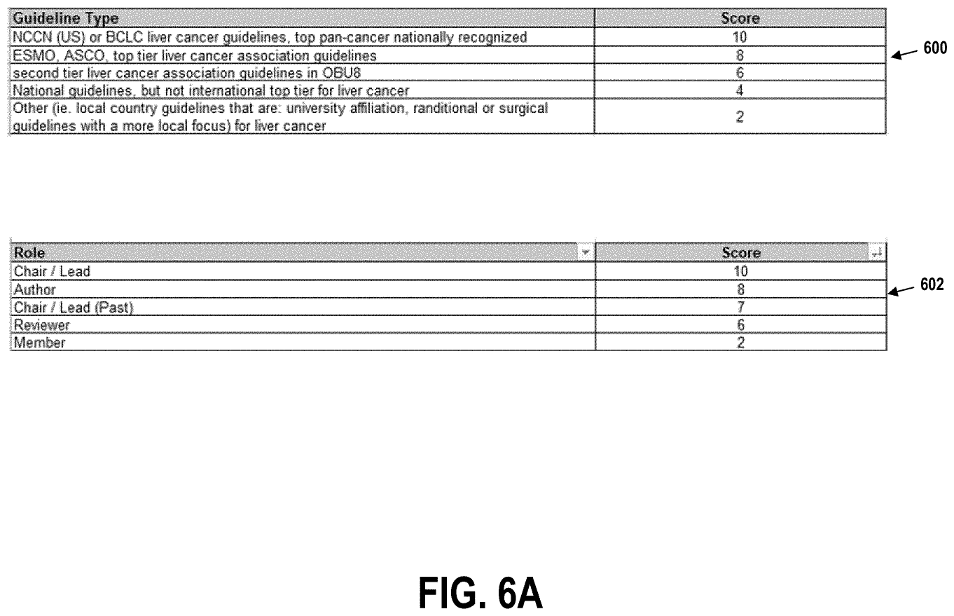

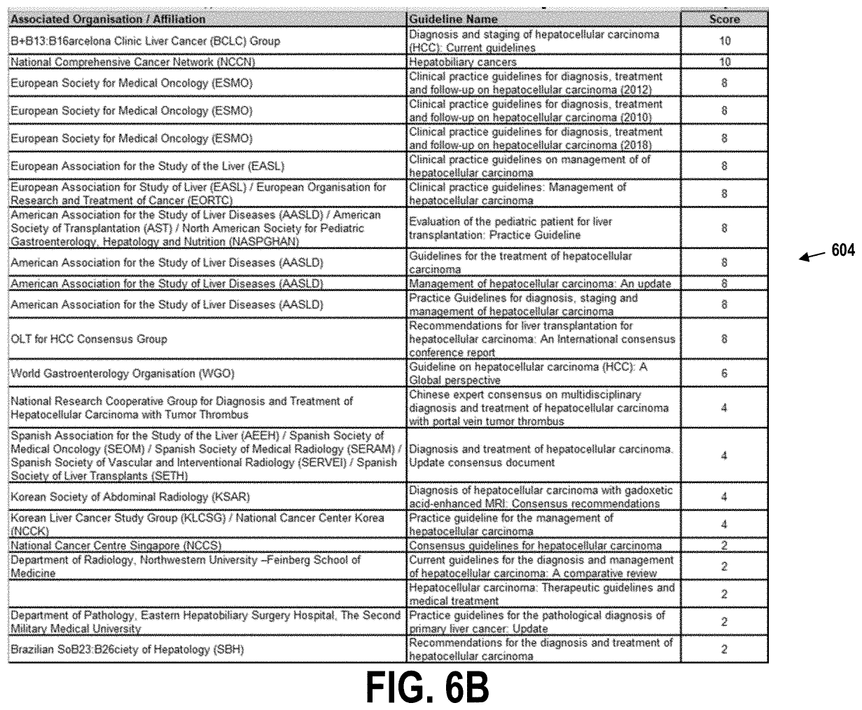

FIG. 6A provides assignments of points to an individual's guidelines activities related to HCC. For the guideline type, as shown in table 600, National Comprehensive Cancer Network (NCCN) or Barcelona Clinic Liver Cancer (BCLC) guidelines, as well as top pan-cancer related guidelines that are nationally recognized, are assigned 10 points. European Society for Medical oncology (ESMO), American Society of Clinical Oncology (ASCO) and other top-tier liver cancer association guidelines are assigned 8 points. Second tier liver cancer association guidelines in OBU8 are assigned 6 points. National guidelines for liver cancer that are not considered top-tier internationally are assigned 4 points. Other guidelines are assigned 2 points.

For the individual's role within development of the guidelines, and as shown in table 602, 10 points are assigned to the chair or lead, 8 points are assigned to an author, 7 points are assigned to a past chair or lead, 6 points are assigned to a reviewer, and 2 points are assigned to a member.

FIG. 6B provides table 604, which maps actual guidelines to assigned point values, in accordance with table 600. As indicated by the conditions of table 600, guidelines published by more prestigious organizations that are more focused on liver cancer are allocated more points, while increasingly fewer points are assigned to guidelines that are less prestigious or are less focused on liver cancer.

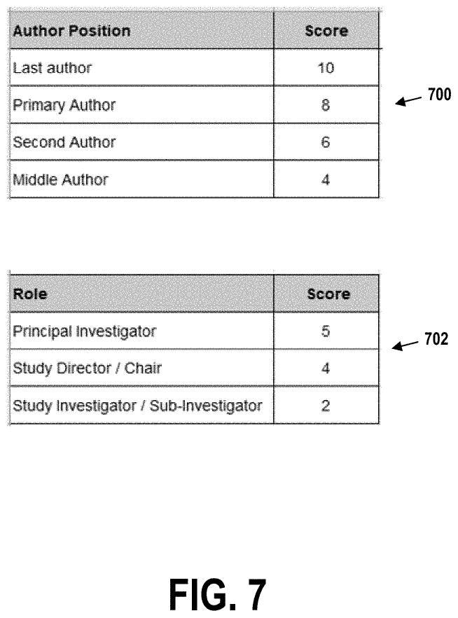

FIG. 7 provides assignments of points to an individual's clinical trial activities related to HCC. For author position, and as shown in table 700, 10 points are assigned to the last author, 8 points are assigned to the primary author, 6 points are assigned to the second author, and 4 points are assigned to the middle author(s). Here, the term “middle author” is defined as was the case for journal publications. For role, and as shown in table 702, 5 points are assigned to the principal investigator, 4 points are assigned to a study director or chair, and 2 points are assigned to a study investigator or sub-investigator.

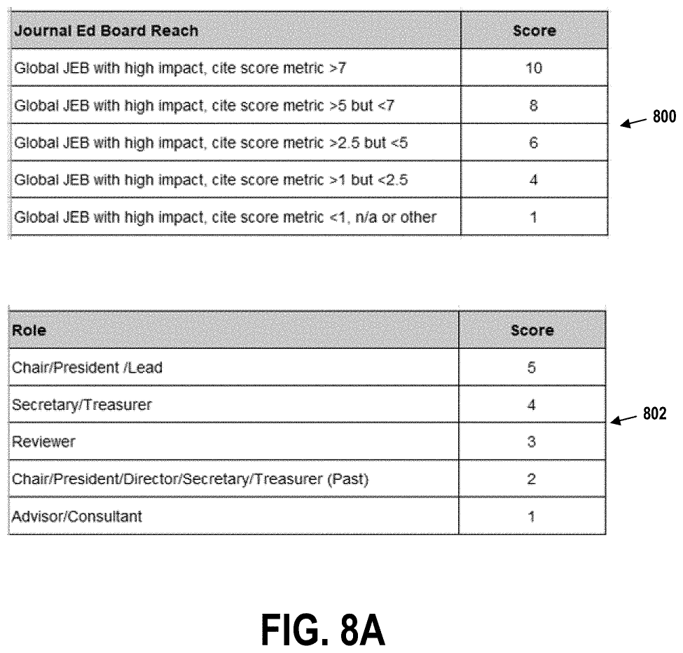

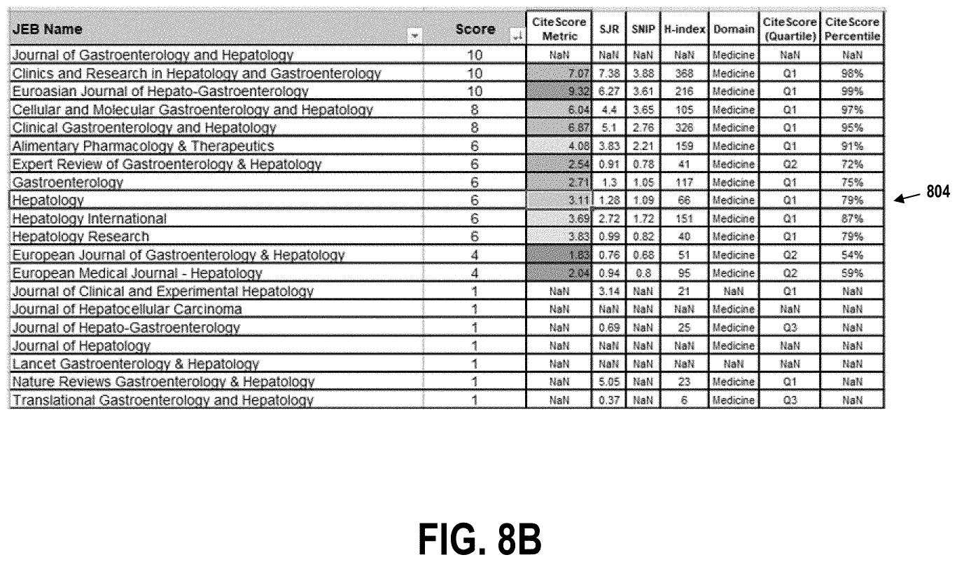

FIG. 8A provides assignments of points to an individual's journal editorial board activities related to HCC. For board reach, and as shown in table 800, global boards with high impact and a cite score above 7 are assigned 10 points. Here, the impact is based on the yearly average number of citations that recent articles published in the journal have received and/or the average citations per article published in the journal over a multi-year period (e.g., 2 years, 3, years, 5 years).

For role, and as shown in table 802, a chair, president or lead is assigned 5 points, a secretary or treasurer is assigned 4 points, a reviewer is assigned 3 points, a past chair, president, director, secretary, or treasurer is assigned 2 points, and an advisor or consultant is assigned 1 point.

FIG. 8B provides table 804, which maps actual journals to assigned point values, in accordance with table 800. As indicated by the conditions of table 800, journals with higher impacts and cite scores are allocated more points, while increasingly fewer points are assigned to journals with lower impacts and/or cite scores.

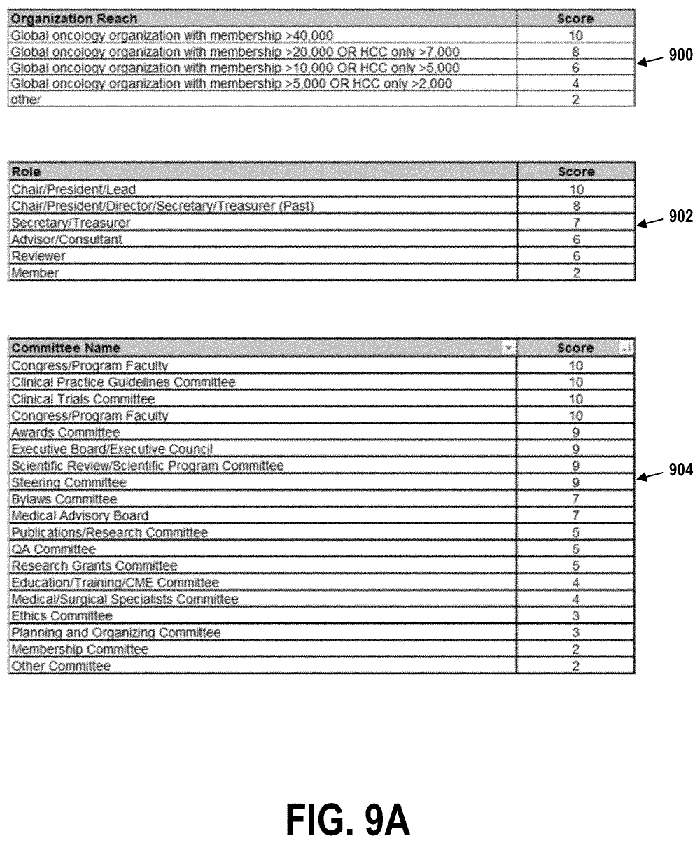

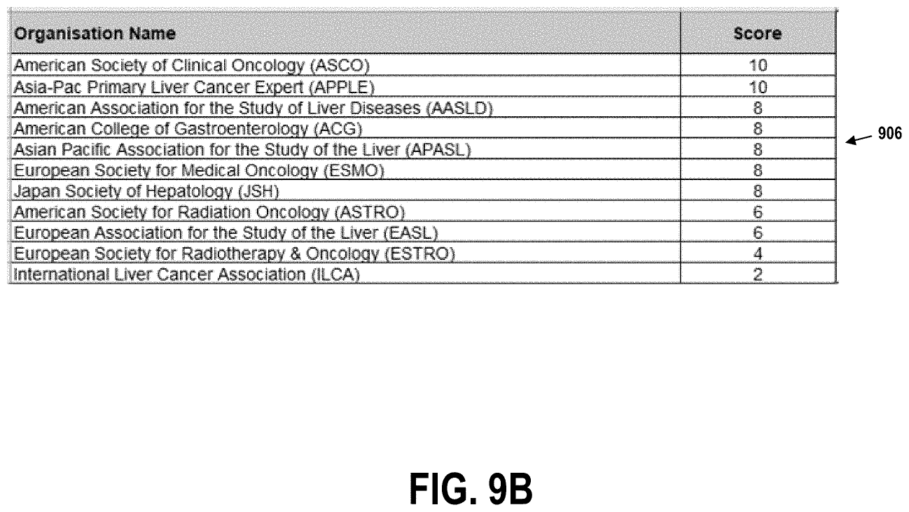

FIG. 9A provides assignments of points to an individual's activities within an organization related to HCC. For organization reach, and as shown in table 900, organizations with over 40,000 members and focused on oncology are assigned 10 points, organizations with over 20,000 members (and up to 40,000 members) focused on oncology or organizations with over 7,000 members focused on HCC are assigned 8 points, organizations with over 10,000 members (and up to 20,000 members) focused on oncology or organizations with over 5,000 members (and up to 7,000 members) focused on HCC are assigned 6 points, organizations with over 5,000 members (and up to 10,000 members) focused on oncology or organizations with over 2,000 members (and up to 5,000 members) focused on HCC are assigned 2 points, and other organizations are assigned 1 point

For role, and as shown in table 902, a chair, president or lead is assigned 10 points, a past chair, president, director, secretary, or treasurer is assigned 8 points, a secretary or treasurer is assigned 7 points, an advisor or consultant is assigned 6 points, a reviewer is assigned 4 points, and a regular member is assigned 2 points.

For committees, and as shown in table 904, those involving organizing activities for congresses, guidelines and clinical trials are assigned the most points. Less scientific and more administrative committees are assigned the least points.

FIG. 9B provides table 906, which maps actual organizations to assigned point values, in accordance with table 900. As indicated by the conditions of table 900, organizations with a larger reach and HCC relevance are allocated more points, while increasingly fewer points are assigned to organizations with lower reaches and/or HCC relevance.

### IV. Example: Non-Small-Cell Lung Carcinoma (NSCLC) and Small Cell Lung Cancer (SCLC)

In order to further illustrate the embodiments herein, specific examples are provided in the sub-sections below for non-small-cell lung cancer (NSCLC) and small cell lung cancer (SCLC), the two main types of lung cancer. Thus, the values assigned to various publications, congresses, guidelines, clinical trials, journal editorial boards, and organizations are based on their prominence and relevance to the study and treatment of these forms of lung cancer. In all of the point assignments below, exactly one point value from each table is selected. If the individual qualifies to be allocated different point values from the same table, the most accurate description of the individual's activity determines the point value. Failing that, the highest or lowest such point value may be selected based on a pre-defined configuration.

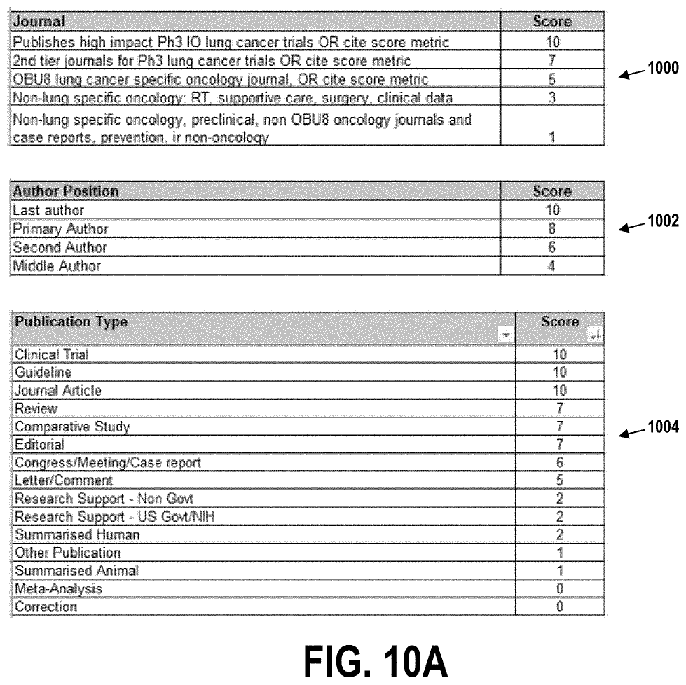

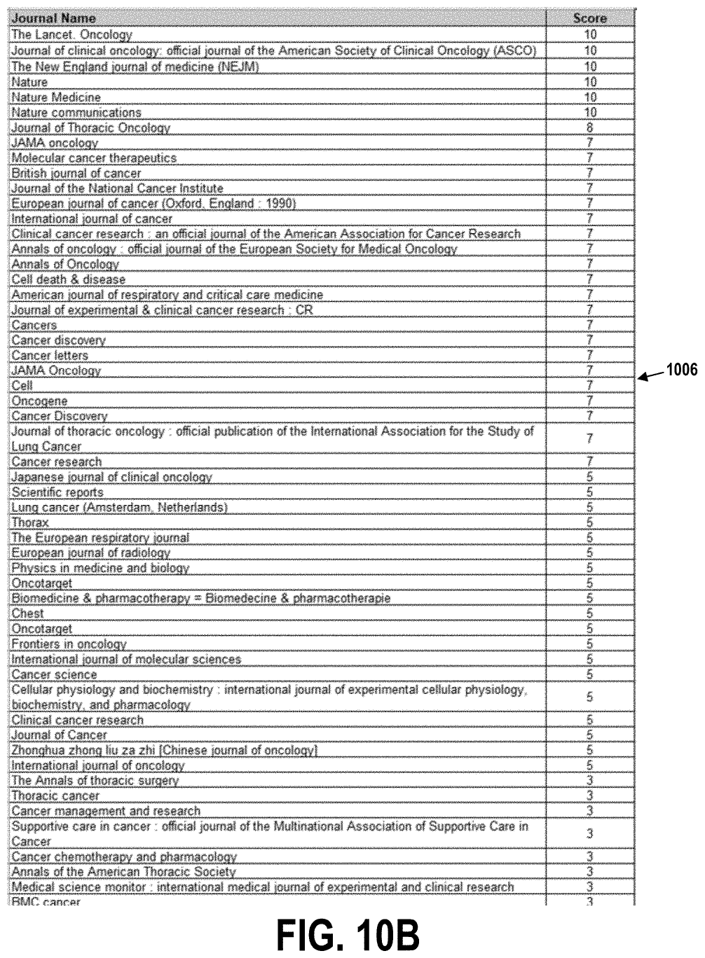

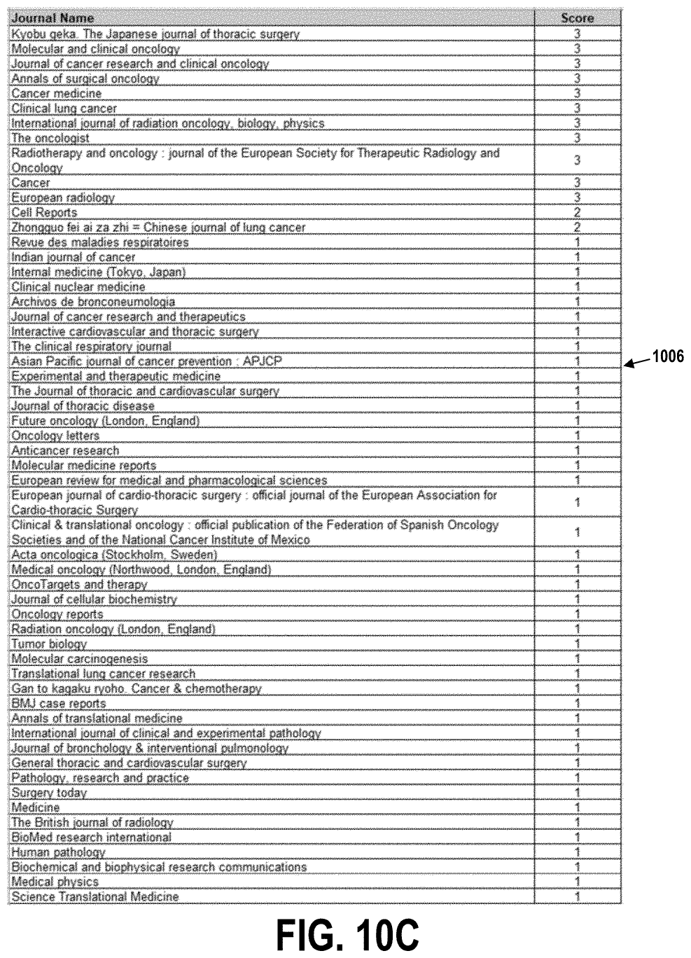

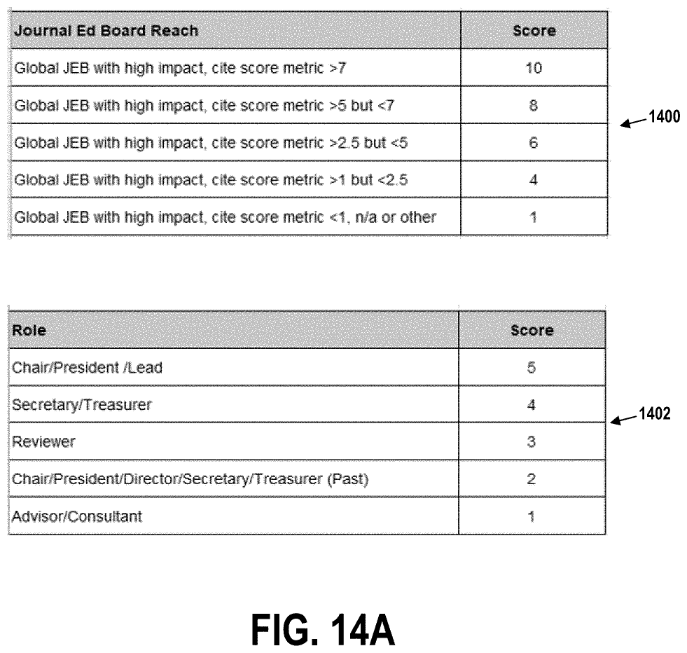

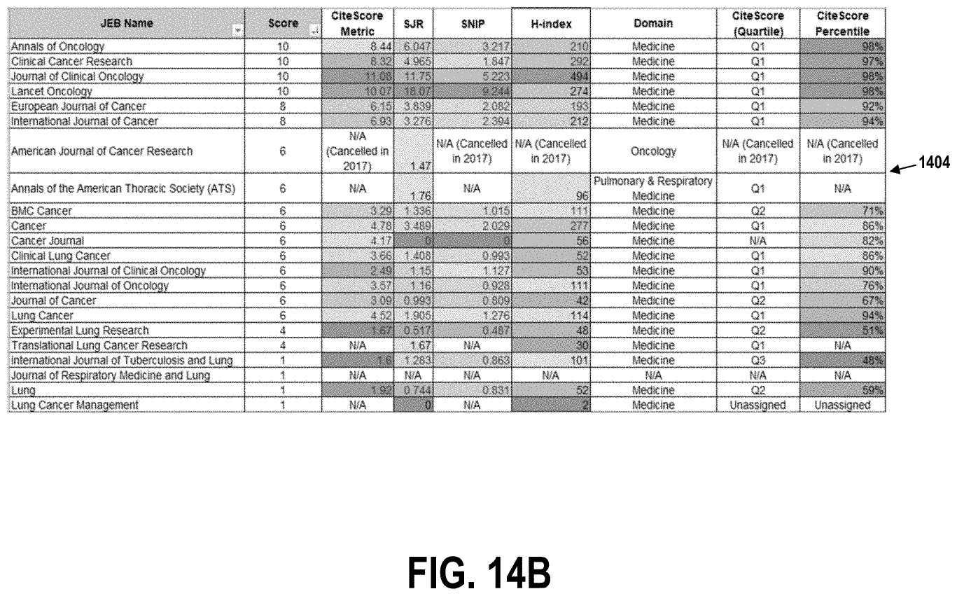

FIG. 10A provides assignments of points to an individual's journal publications related to lung cancer. For journals, and as shown in table 1000, 10 points are assigned to journals that publish high impact phase 3 clinical data in lung cancer trials or those that have a sufficiently high cite score metric, 7 points are assigned to second-tier journals for phase 3 clinical data in lung cancer trials or those that have a sufficiently high cite score metric, 5 points are assigned to OBU8 lung cancer oncology journals or those that have a sufficiently high cite score metric, 3 points are assigned to non-lung oncology journals focused on radiation therapy, supportive care, or surgery, and 1 point is assigned to non-lung, preclinical and non-OBU8 oncology journals focused on preclinical studies, case reports, or prevention. As noted above, the cite score is based on the average citations per document (e.g., paper, letter, etc.) that a journal receives over a three-year period. Examples of cite score metrics for journals are provided in FIG. 14B.

For author position, and as shown in table 1002, 10 points are assigned to the last author, 8 points are assigned to the primary author, 6 points are assigned to the second author, and 4 points are assigned to the middle author(s). Here, a “middle author” is any author positioned in other than the first, second, or last position. For example, on a paper with five authors, the authors in the third and fourth positions are both middle authors.

For publication type, and as shown in table 1004, 10 points are assigned to papers on clinical trials, papers on guidelines, and journal articles. Other types of publications are assigned increasingly fewer points. For example, meta-analyses and corrections are assigned 0 points. As noted above, double-counting of such papers is possible but acceptable.

The lowest score for a listed author is 5 points. This occurs when the author is a middle author (4 points), the journal is in the non-lung, preclinical and non-OBU8 oncology category (1 point), and the type of publication is meta-analysis or correction (0 points).

FIGS. 10B and 10C provide table 1006, which maps actual journals to assigned point values, in accordance with table 1000. As indicated by the conditions of table 1000, the journals assigned 10 points are assigned to prestigious journals that focus on lung cancer, while increasingly fewer points are assigned to journals that are less prestigious or are less focused on lung cancer.

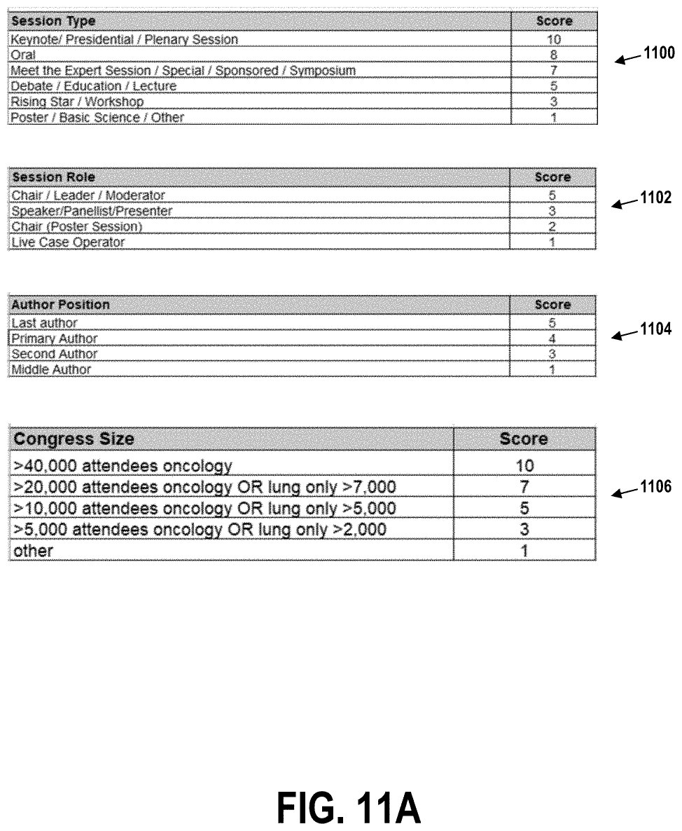

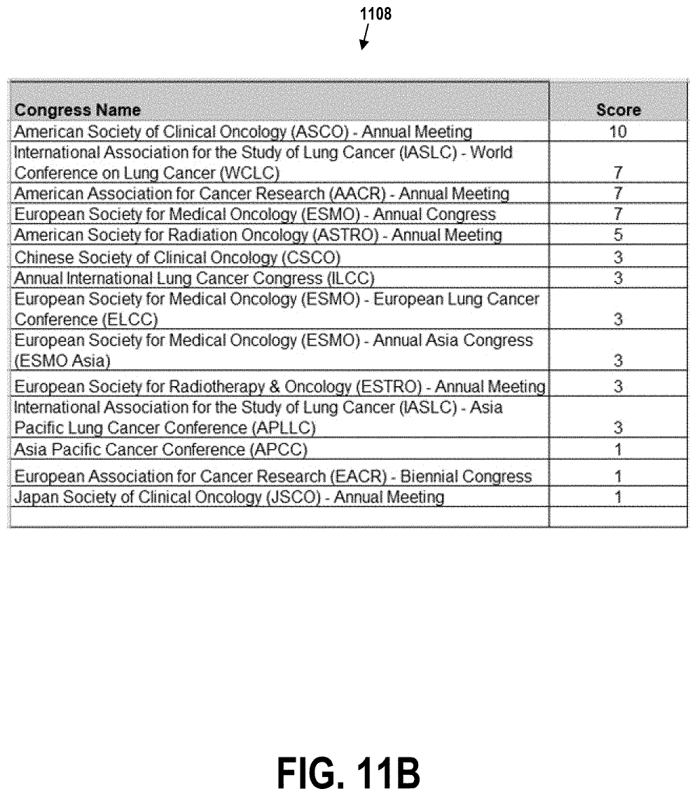

FIG. 11A provides assignments of points to an individual's congress activities related to lung cancer. For the session type, and as shown in table 1100, keynote, presidential and plenary sessions are assigned 10 points, other oral presentations are assigned 8 points, meet the experts sessions, special sessions, sponsored sessions, and symposia are assigned 7 points, debates, educational sessions, and lectures are assigned 5 points, rising star sessions and workshops are assigned 3 points, and posters, basic science sessions other types of sessions are assigned 1 point.

For the individual's role within the session, and as shown in table 1102, chairs, leaders, and moderators are assigned 5 points, speakers, panelists, and presenters and assigned 3 points, chairs of poster sessions are assigned 2 points, and live case operators are assigned 1 point.

For author position, and as shown in table 1104, 5 points are assigned to the last author, 4 points are assigned to the primary author, 3 points are assigned to the second author, and 1 point is assigned to the middle author(s). Here, the term “middle author” is defined as was the case for journal publications.

For congress size, and as shown in table 1106, congresses with over 40,000 attendees and focused on oncology are assigned 10 points, congresses with over 20,000 attendees (and up to 40,000 attendees) focused on oncology or congresses with over 7,000 attendees focused on lung cancer are assigned 7 points, congresses with over 10,000 attendees (and up to 20,000 attendees) focused on oncology or congresses with over 5,000 attendees (and up to 7,000 attendees) focused on lung cancer are assigned 5 points, congresses with over 5,000 attendees (and up to 10,000 attendees) focused on oncology or congresses with over 2,000 attendees (and up to 5,000 attendees) focused on lung cancer are assigned 3 points, and other congresses are assigned 1 point.

FIG. 11B provides table 1108, which maps actual congresses to assigned point values, in accordance with table 1106. As indicated by the conditions of table 1106, larger congresses that are more focused on lung cancer are allocated more points, while increasingly fewer points are assigned to congresses that are smaller or are less focused on lung cancer.

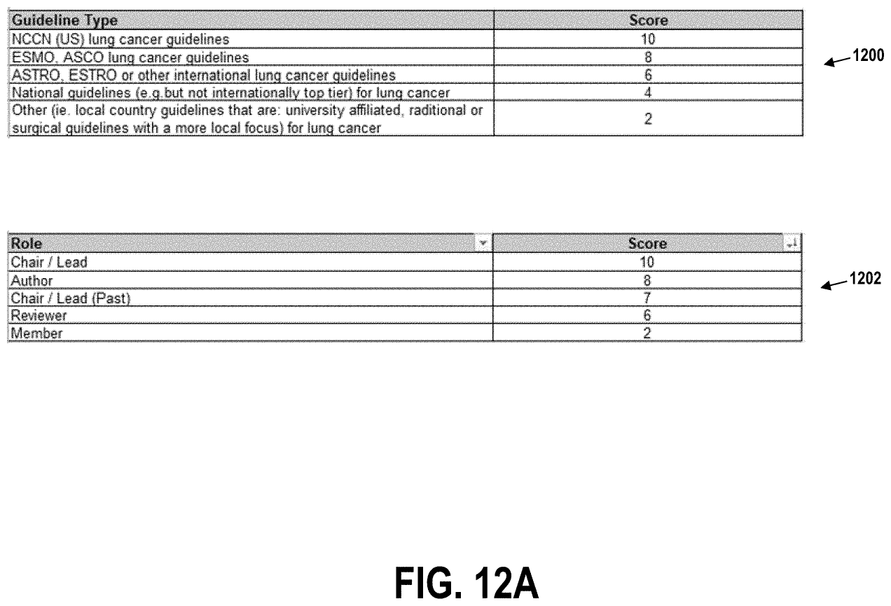

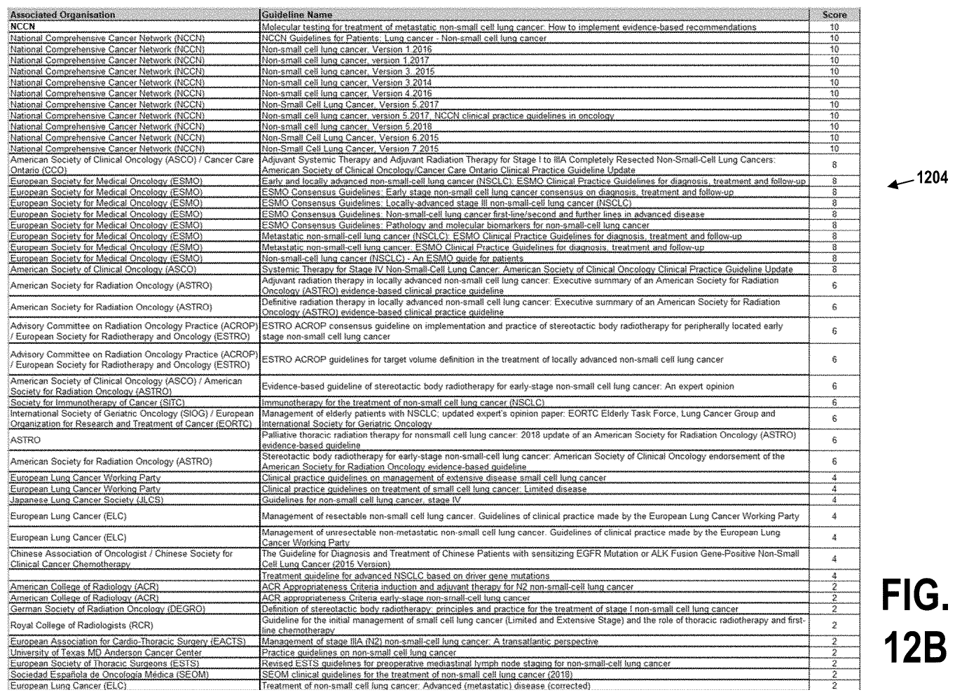

FIG. 12A provides assignments of points to an individual's guidelines activities related to lung cancer. For the guideline type, as shown in table 1200, National Comprehensive Cancer Network (NCCN) guidelines are assigned 10 points. European Society for Medical oncology (ESMO) and American Society of Clinical Oncology (ASCO) guidelines are assigned 8 points. American Society for Radiation Oncology (ASTRO) and European Society for Radiotherapy and Oncology (ESTRO), as well as other international lung cancer guidelines are assigned 6 points. National guidelines for lung cancer that are not considered top-tier internationally are assigned 4 points. Other guidelines are assigned 2 points.

For the individual's role within development of the guidelines, and as shown in table 1202, 10 points are assigned to the chair or lead, 8 points are assigned to an author, 7 points are assigned to a past chair or lead, 6 points are assigned to a reviewer, and 2 points are assigned to a member.

FIG. 12B provides table 1204, which maps actual guidelines to assigned point values, in accordance with table 1200. As indicated by the conditions of table 1200, guidelines published by more prestigious organizations that are more focused on lung cancer are allocated more points, while increasingly fewer points are assigned to guidelines that are less prestigious or are less focused on lung cancer.

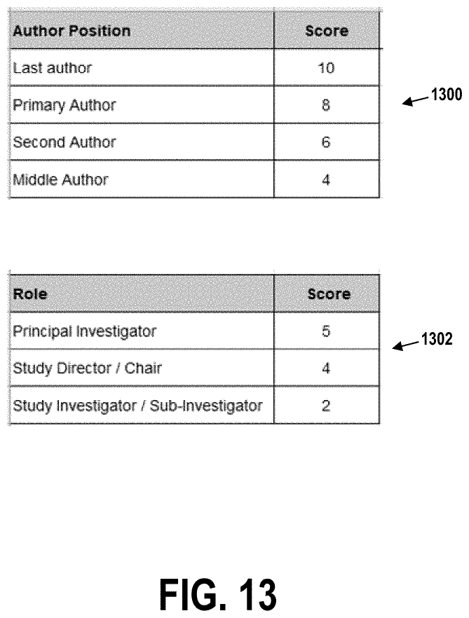

FIG. 13 provides assignments of points to an individual's clinical trial activities related to lung cancer. For author position, and as shown in table 1300, 10 points are assigned to the last author, 8 points are assigned to the primary author, 6 points are assigned to the second author, and 4 points are assigned to the middle author(s). Here, the term “middle author” is defined as above. For role, and as shown in table 1302, 5 points are assigned to the principal, 4 points are assigned to a study director or chair, and 2 points are assigned to a study investigator or sub-investigator.

FIG. 14A provides assignments of points to an individual's journal editorial board activities related to lung cancer. For board reach, and as shown in table 1400, global boards with high impact and a cite score above 7 are assigned 10 points. Here, the impact is based on the yearly average number of citations that recent articles published in the journal have received and/or the average citations per article published in the journal over a multi-year period (e.g., 2 years, 3, years, 5 years).

For role, and as shown in table 1402, a chair, president or lead is assigned 5 points, a secretary or treasurer is assigned 4 points, a reviewer is assigned 3 points, a past chair, president, director, secretary, or treasurer is assigned 2 points, and an advisor or consultant is assigned 1 point.

FIG. 14B provides table 1404, which maps actual journals to assigned point values, in accordance with table 1400. As indicated by the conditions of table 1400, journals with higher impacts and cite scores are allocated more points, while increasingly fewer points are assigned to journals with lower impacts and/or cite scores.

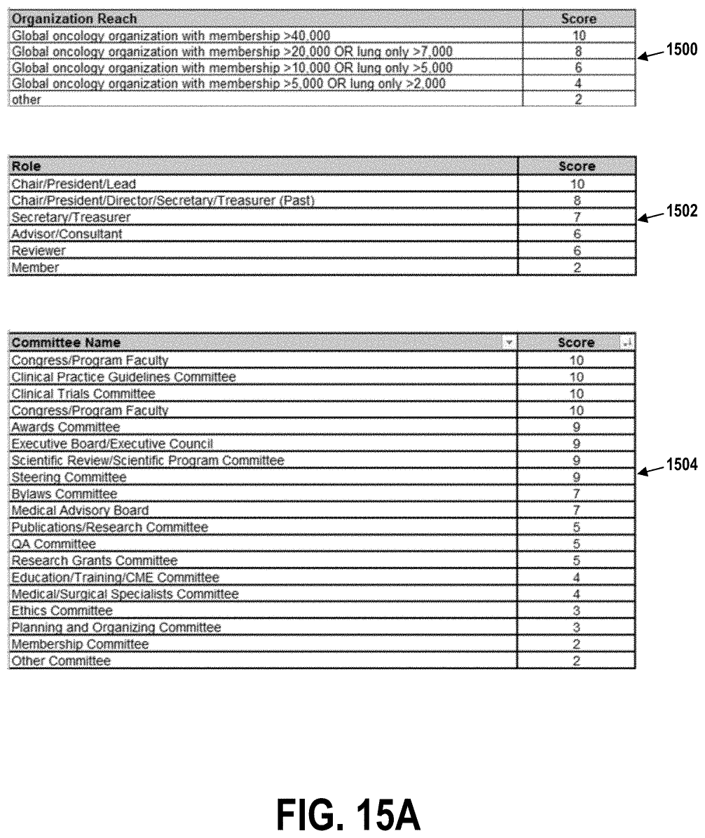

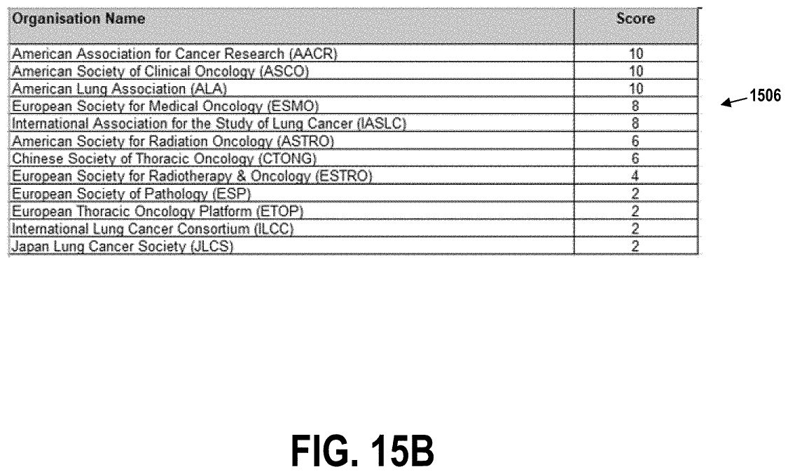

FIG. 15A provides assignments of points to an individual's activities within an organization related to lung cancer. For organization reach, and as shown in table 1500, organizations with over 40,000 members and focused on oncology are assigned 10 points, organizations with over 20,000 members (and up to 40,000 members) focused on oncology or organizations with over 7,000 members focused on lung cancer are assigned 8 points, organizations with over 10,000 members (and up to 20,000 members) focused on oncology or organizations with over 5,000 members (and up to 7,000 members) focused on lung cancer are assigned 6 points, organizations with over 5,000 members (and up to 10,000 members) focused on oncology or organizations with over 2,000 members (and up to 5,000 members) focused on lung cancer are assigned 2 points, and other organizations are assigned 1 point

For role, and as shown in table 1502, a chair, president or lead is assigned 10 points, a past chair, president, director, secretary, or treasurer is assigned 8 points, a secretary or treasurer is assigned 7 points, an advisor or consultant is assigned 6 points, a reviewer is assigned 4 points, and a regular member is assigned 2 points.

For committees, and as shown in table 1504, those involving organizing activities for congresses, guidelines and clinical trials are assigned the most points. Less scientific and more administrative committees are assigned the least points.

FIG. 15B provides table 1506, which maps actual organizations to assigned point values, in accordance with table 1500. As indicated by the conditions of table 1500, organizations with a larger reach and lung cancer relevance are allocated more points, while increasingly fewer points are assigned to organizations with lower reaches and/or lung cancer relevance.

### V. Uses and Advantages

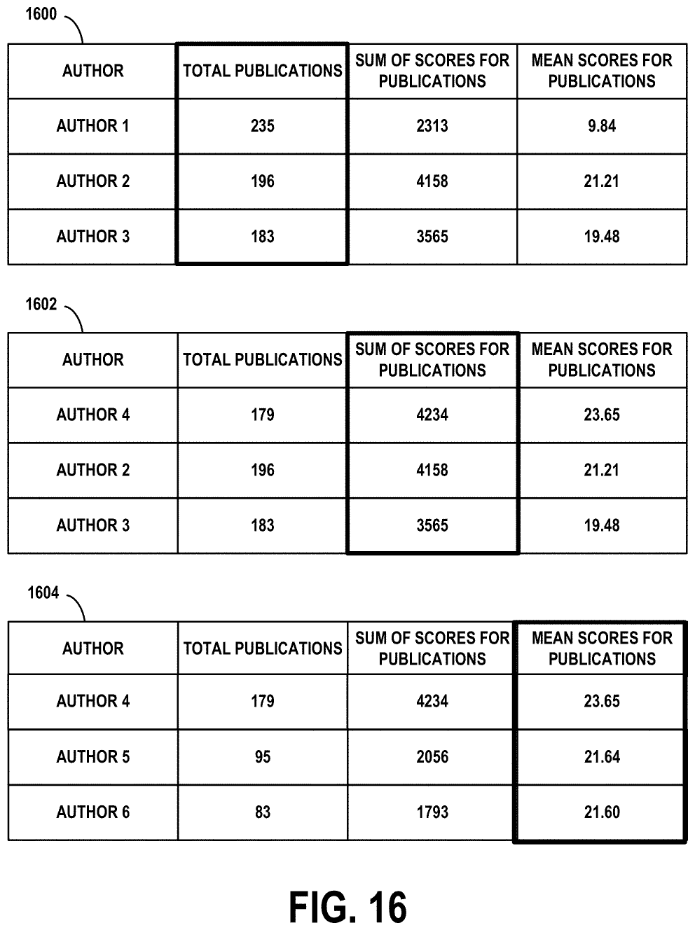

FIG. 16 provides three tables that illustrate uses and advantages of the embodiments discussed above. These three tables focus only on the publications score, but can readily be generalized for any of the other scores.

As notes above, the publications score for each individual takes on a value between 0 and 30 per publication, with higher scores generally being more prestigious, relevant, and impactful for a given medical or scientific specialty. Using this score rather than a raw count of publications has been found to result in a more accurate assessment of individuals' expertise in that specialty.

For instance, table 1600 show three metrics for three hypothetical authors. The first metric is total publications. For purposes of this discussion, this is a simple count of each author's total number of journal publications, regardless of journal and regardless of type.

The second metric is the sum of scores for publications of each author. This metric is calculated using the publications metrics above and results in a score of 0-30 per publication per author. These scores are summed to provide the sum of scores for publications. For example, author 2 has 196 publications and a sum of scores of 4158.

The third metric is mean score for publications. This metric is the second metric divided by the first metric; that is, the sum of scores of publications divided by the total publications. Accordingly, the third metric will always be between 0 and 30 inclusive.

Depending on which metric is considered, a different set of authors may be ranked highest. In table 1600, authors are ranked by total publications, which measures the volume of publications per author. According to this metric, author 1 is first, author 2 is second, and author 3 is third.

In table 1602, authors are ranked by the sum of scores of publications, which measures both volume and impact of publications per author. According to this metric, author 4 is first. Notably, author 4 did not appear in the top three authors ranked according to metric 1.

In table 1604, authors are ranked by the mean scores for publications, which measures the impact of publications per author regardless of volume. According to this metric, author 4 is first, followed by authors 5 and 6. Notably, none of these authors appeared in the top three authors ranked according to metric 1, and authors 5 and 6 did not appear in the top 3 authors ranked according to metric 2.

### VI. Example Operations

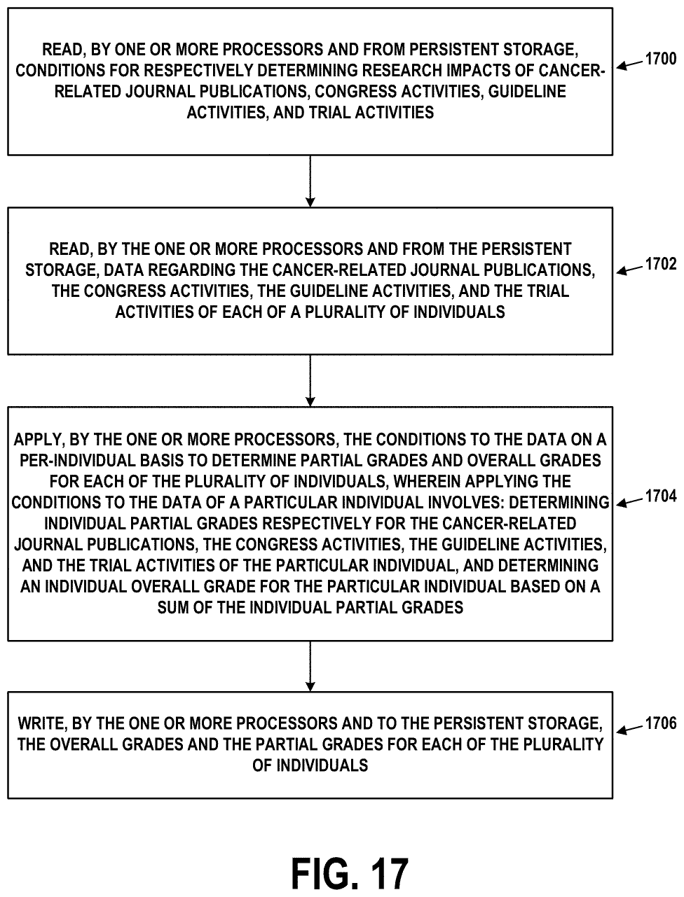

FIG. 17 is a flow chart illustrating an example embodiment. The process illustrated by FIG. 17 may be carried out by a computing device, such as computing device 100, and/or a cluster of computing devices, such as server cluster 200 or computational instance 322. However, the process can be carried out by other types of devices or device subsystems.

The embodiments of FIG. 17 may be simplified by the removal of any one or more of the features shown therein. Further, these embodiments may be combined with features, aspects, and/or implementations of any of the previous figures or otherwise described herein.

Block 1700 may involve reading, by one or more processors and from persistent storage, conditions for respectively determining research impacts of cancer-related journal publications, congress activities, guideline activities, and trial activities.

Block 1702 may involve reading, by the one or more processors and from the persistent storage, data regarding the cancer-related journal publications, the congress activities, the guideline activities, and the trial activities of each of a plurality of individuals.

Block 1704 may involve applying, by the one or more processors, the conditions to the data on a per-individual basis to determine partial grades and overall grades for each of the plurality of individuals, wherein applying the conditions to the data of a particular individual involves: determining individual partial grades respectively for the cancer-related journal publications, the congress activities, the guideline activities, and the trial activities of the particular individual, and determining an individual overall grade for the particular individual based on a sum of the individual partial grades.

Block 1706 may involve writing, by the one or more processors and to the persistent storage, the overall grades and the partial grades for each of the plurality of individuals.

In some embodiments, the conditions for determining the research impacts of the cancer-related journal publications include titles of cancer-related journals, author positions in the cancer-related journal publications, and types of the cancer-related journal publications.

In some embodiments, the conditions for determining the research impacts of the cancer-related congress activities include session types within cancer-related congresses, session roles within the cancer-related congresses, author positions in publications of the cancer-related congresses, and congress sizes for the cancer-related congresses.

In some embodiments, the conditions for determining the research impacts of the cancer-related guideline activities include types of cancer-related guidelines and roles of in developing the cancer-related guidelines.

In some embodiments, the conditions for determining the research impacts of cancer-related trial activities include author positions in publications of the cancer-related trial activities and roles in the cancer-related trial activities.

In some embodiments, the conditions are also for determining research impact of cancer-related journal editorial board activities, the data also includes the cancer-related journal editorial board activities of each of the plurality of individuals, wherein one of the partial grades is for the cancer-related journal editorial board activities. In these embodiments, the conditions for determining the research impacts of cancer-related journal editorial board activities include reach of a cancer-related journal and roles in the cancer-related journal editorial board activities.

In some embodiments, the conditions are also for determining research impact of cancer-related organization activities, wherein the data also includes the cancer-related organization activities of each of the plurality of individuals, wherein one of the partial grades is for the cancer-related organization activities. In these embodiments, the conditions for determining the research impacts of the cancer-related organization activities include reach of a cancer-related organization, roles in the cancer-related organization activities, and committees associated with the cancer-related organization activities.

In some embodiments, each of the partial grades is represented on a scale of 0-30, with higher values indicating higher impact or relevance. In these embodiments, each of the overall grades is a sum of an average of corresponding partial grades of one of the individuals and is represented on a scale of 0-180.

Some embodiments may further involve: sorting, from highest to lowest, the plurality of individuals by the partial grades or the overall grades; and providing, for display on a client device, a listing of the individuals as sorted with their corresponding partial grades or overall grades. In these embodiments, providing the listing of the individuals as sorted with their corresponding partial grades or overall grades comprises providing a representation of a graphical user interface including: (i) a first set of columns for identifying the individuals, and (ii) a second set of columns for identifying their corresponding partial grades or overall grades. Moreover, each column in the first set of columns and the second set of columns may be sortable by way of the graphical user interface.

### VII. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid-state drive, or another storage medium.

The computer readable medium can also include non-transitory computer readable media such as computer readable media that store data for short periods of time like register memory and processor cache. The computer readable media can further include non-transitory computer readable media that store program code and/or data for longer periods of time. Thus, the computer readable media may include secondary or persistent long-term storage, like ROM, optical or magnetic disks, solid-state drives, compact-disc read only memory (CD-ROM), for example. The computer readable media can also be any other volatile or non-volatile storage systems. A computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A computer-implemented method comprising:
reading, by one or more processors and from persistent storage, conditions for respectively determining research impacts of cancer-related journal publications, congress activities, guideline activities, and trial activities;
reading, by the one or more processors and from the persistent storage, data regarding the cancer-related journal publications, the congress activities, the guideline activities, and the trial activities of each of a plurality of individuals;
applying, by the one or more processors, the conditions to the data on a per-individual basis to determine partial grades and overall grades for each of the plurality of individuals, wherein applying the conditions to the data of a particular individual involves: determining individual partial grades respectively for the cancer-related journal publications, the congress activities, the guideline activities, and the trial activities of the particular individual, and determining an individual overall grade for the particular individual based on a sum of the individual partial grades; and
writing, by the one or more processors and to the persistent storage, the overall grades and the partial grades for each of the plurality of individuals.

2. The computer-implemented method of claim 1, wherein the conditions for determining the research impacts of the cancer-related journal publications include titles of cancer-related journals, author positions in the cancer-related journal publications, and types of the cancer-related journal publications.

3. The computer-implemented method of claim 1, wherein the conditions for determining the research impacts of the cancer-related congress activities include session types within cancer-related congresses, session roles within the cancer-related congresses, author positions in publications of the cancer-related congresses, and congress sizes for the cancer-related congresses.

4. The computer-implemented method of claim 1, wherein the conditions for determining the research impacts of the cancer-related guideline activities include types of cancer-related guidelines and roles of in developing cancer-related guidelines.

5. The computer-implemented method of claim 1, wherein the conditions for determining the research impacts of cancer-related trial activities include author positions in publications of the cancer-related trial activities and roles in the cancer-related trial activities.

6. The computer-implemented method of claim 1, wherein the conditions are also for determining research impact of cancer-related journal editorial board activities, wherein the data also includes the cancer-related journal editorial board activities of each of the plurality of individuals, wherein one of the partial grades is for the cancer-related journal editorial board activities.

7. The computer-implemented method of claim 6, wherein the conditions for determining the research impacts of cancer-related journal editorial board activities include reach of a cancer-related journal and roles in the cancer-related journal editorial board activities.

8. The computer-implemented method of claim 1, wherein the conditions are also for determining research impact of cancer-related organization activities, wherein the data also includes the cancer-related organization activities of each of the plurality of individuals, wherein one of the partial grades is for the cancer-related organization activities.

9. The computer-implemented method of claim 8, wherein the conditions for determining the research impacts of the cancer-related organization activities include reach of a cancer-related organization, roles in the cancer-related organization activities, and committees associated with the cancer-related organization activities.

10. The computer-implemented method of claim 1, wherein each of the partial grades is represented on a scale of 0-30, with higher values indicating higher impact or relevance.

11. The computer-implemented method of claim 10, wherein each of the overall grades is a sum of an average of corresponding partial grades of one of the individuals and is represented on a scale of 0-180.

12. The computer-implemented method of claim 1, further comprising:
sorting, from highest to lowest, the plurality of individuals by the partial grades or the overall grades; and
providing, for display on a client device, a listing of the individuals as sorted with their corresponding partial grades or overall grades.

13. The computer-implemented method of claim 12, wherein providing the listing of the individuals as sorted with their corresponding partial grades or overall grades comprises providing a representation of a graphical user interface including: (i) a first set of columns for identifying the individuals, and (ii) a second set of columns for identifying their corresponding partial grades or overall grades.

14. The computer-implemented method of claim 13, wherein each column in the first set of columns and the second set of columns is sortable by way of the graphical user interface.

15. An article of manufacture including a non-transitory computer-readable medium, having stored thereon program instructions that, upon execution by a computing device, cause the computing device to perform operations comprising:
reading, from persistent storage, conditions for respectively determining research impacts of cancer-related journal publications, congress activities, guideline activities, and trial activities;
reading, from the persistent storage, data regarding the cancer-related journal publications, the congress activities, the guideline activities, and the trial activities of each of a plurality of individuals;
applying the conditions to the data on a per-individual basis to determine partial grades and overall grades for each of the plurality of individuals, wherein applying the conditions to the data of a particular individual involves: determining individual partial grades respectively for the cancer-related journal publications, the congress activities, the guideline activities, and the trial activities of the particular individual, and determining an individual overall grade for the particular individual based on a sum of the individual partial grades; and
writing, to the persistent storage, the overall grades and the partial grades for each of the plurality of individuals.

16. The article of manufacture of claim 15, wherein each of the partial grades is represented on a scale of 0-30, with higher values indicating higher impact or relevance.

17. The article of manufacture of claim 16, wherein each of the overall grades is a sum of an average of corresponding partial grades of one of the individuals and is represented on a scale of 0-180.

18. The article of manufacture of claim 15, the operations further comprising:
sorting, from highest to lowest, the plurality of individuals by the partial grades or the overall grades; and
providing, for display on a client device, a listing of the individuals as sorted with their corresponding partial grades or overall grades.

19. The article of manufacture of claim 18, wherein providing the listing of the individuals as sorted with their corresponding partial grades or overall grades comprises providing a representation of a graphical user interface including: (i) a first set of columns for identifying the individuals, and (ii) a second set of columns for identifying their corresponding partial grades or overall grades.

20. A computing device comprising:
a processor;
persistent storage; and
program instructions, stored in the persistent storage, that upon execution by the processor cause the computing device to perform operations comprising:
reading, from persistent storage, conditions for respectively determining research impacts of cancer-related journal publications, congress activities, guideline activities, and trial activities;
reading, from the persistent storage, data regarding the cancer-related journal publications, the congress activities, the guideline activities, and the trial activities of each of a plurality of individuals;
applying the conditions to the data on a per-individual basis to determine partial grades and overall grades for each of the plurality of individuals, wherein applying the conditions to the data of a particular individual involves: determining individual partial grades respectively for the cancer-related journal publications, the congress activities, the guideline activities, and the trial activities of the particular individual, and determining an individual overall grade for the particular individual based on a sum of the individual partial grades; and
writing, to the persistent storage, the overall grades and the partial grades for each of the plurality of individuals.
